(19) United States
(12) Patent Application Publication
(10) Pub. No.: US 2013/0034496 A1
(43) Pub. Date: Feb. 7, 2013

(54) POSITRON EMISSION TOMOGRAPHY TRACER

(71) Applicant: Amnon Kohen, Iowa City, IA (US)

(72) Inventor: Amnon Kohen, Iowa City, IA (US)

(73) Assignee: UNIVERSITY OF IOWA RESEARCH FOUNDATION, Iowa City, IA (US)

(21) Appl. No.: 13/565,599

(22) Filed: Aug. 2, 2012

Related U.S. Application Data
(60) Provisional application No. 61/515,271, filed on Aug. 4, 2011.

(51) Int. Cl. A61K 51/04; C12Q 1/16; C07D 475/04

(52) U.S. Cl. 424/1.81; 544/258; 435/35

(57) ABSTRACT

The present invention relates to positron emission tomography tracers and methods of using these tracers.

### PRIORITY OF INVENTION

This application claims priority to U.S. Provisional Application No. 61/515,271 that was filed on Aug. 4, 2011. The entire content of this provisional application is hereby incorporated herein by reference.

### BACKGROUND OF THE INVENTION

Molecular imaging technologies are widely used clinical tools for the diagnosis, staging, and monitoring or therapeutic responses of cancer. Many different technologies have been developed to image the structure and function of systems such as autoradiography, optical imaging, positron emission tomography (PET), magnetic resonance imaging (MRI), and X-ray computed tomography (CT). Among those, PET is the only non-invasive technology that can measure metabolic, biochemical and functional activity in vivo. Since morphological response to chemotherapy or radiation therapy lags behind the course of the treatment, analysis of PET images can potentially detect pathological features and therapeutic response before they are visible on CT and MRI images, and thus PET is emerging as a valuable clinical tool to monitor therapeutic responses in patients.

PET imaging requires positron emitting radioisotopes such as oxygen (¹⁴O, ¹⁵O), nitrogen (¹³N), fluorine-18 (¹⁸F), and Carbon (¹¹C) incorporated into pharmaceutical probes to observe selective accumulation in a tissue of interest. Two of the most extensively used PET probes for cancer are ¹⁸F-fluorodeoxyglucose (¹⁸F-FDG) and ¹⁸F-fluorothymidine (¹⁸F-FLT). The ¹⁸F-FDG probe targets metabolic activity in a non-specific way resulting in high background labeling of normal tissues such as brain and areas of inflammation. On the other hand ¹⁸F-FLT is a proliferation marker and targets the thymidylate kinase 1, which is a scavenging pathway used by some cells when dTMP is required for DNA synthesis. The ¹⁸F-FLT activity in tumor is not always reliable in the detection of viable residuals in patients with viable carcinoma or mature teratoma in histology. False negative and false positive rates for these probes limit their accuracy in monitoring cancer therapy. Accordingly, improved probes and imaging methods are needed.

### SUMMARY OF THE INVENTION

The present inventors have developed an improved PET proliferative tracer with different uptake and/or therapeutic response prediction. Accordingly, in certain embodiments, the present invention provides a compound of [11-¹¹C]—(R)—N⁵,N¹⁰-methylene-5,6,7,8-tetrahydrofolate (¹¹C-MTHF). In certain embodiments, the present invention provides a compound of [11-¹⁴C]—(R)—N⁵,N¹⁰-methylene-5,6,7,8-tetrahydrofolate (¹⁴C-MTHF). In certain embodiments, the present invention provides a compound of[¹¹C]-leucovorin (folinic acid). In certain embodiments, the present invention provides a compound of [¹⁴]-leucovorin (folinic acid). In certain embodiments, the present invention provides a compound of 5-[¹¹C]-methyl-THF. In certain embodiments, the present invention provides a compound of 5-[¹⁴C]-methyl-THF.

In certain embodiments, the present invention further provides a composition comprising the compound described above and a pharmaceutically acceptable carrier.

In certain embodiments, the present invention provides a pharmaceutical composition comprising a diagnostically effective amount of the compound described above, or a pharmaceutically acceptable addition salt thereof, together with at least one pharmaceutically acceptable carrier or diluent.

In cases where compounds are sufficiently basic or acidic, a salt of a compound of the invention can be useful as an intermediate for isolating or purifying a compound of the invention. Additionally, administration of a compound of the invention as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

In certain embodiments, the present invention provides a method of detecting a cancer cell comprising contacting the compound as described above to the cell, and imaging the cell by means of positron emission tomography (PET), wherein the compound is employed as a tracer. In certain embodiments, the cell comprises a folate receptor (FR). In certain embodiments, the FR is FR-alpha. In certain embodiments, the method further comprises the steps of imaging the subject to detect the distribution of folate receptor. In certain embodiments, the method further comprises the steps of imaging the subject and analyzing the imaging data. In certain embodiments, the radiotracer is detected by imaging the subject, the method further comprising the steps of administering to the subject a therapeutic agent; administering to the subject the radiotracer; imaging the subject; and comparing a level of association of the tracer with the folate receptor in the subject before and after administering the therapeutic agent. In certain embodiments, the imaging in the subject is performed by positron emission tomography, single photon emission computed tomography or a combination thereof.

In certain embodiments, the present invention provides a method for in vivo determination of a potential anti-cancer treatment in a subject, comprising i) visualising potential cancer cells with positron emission tomography (PET) or Single Photon Emission Computed Tomography (SPECT), wherein a compound as described above is used as a tracer; ii) administering the potential anti-cancer treatment to the subject; and iii) repeating step i) to determine the effectiveness of the potential anti-cancer treatment.

In certain embodiments, the present invention provides a method for the non-invasive determination of the distribution of a tracer compound inside a whole, intact living animal or human body using a physical detection method, wherein the tracer compound is a compound as described above. In certain embodiments, the compound is measured by Positron Emission Tomography (PET) or Single Photon Emission Computed Tomography (SPECT).

In certain embodiments, the present invention provides a method for in vivo determination of a potential anti-cancer treatment's effectiveness in a subject, comprising i) visualising potential cancer cells with positron emission tomography (PET) or Single Photon Emission Computed Tomography (SPECT), wherein a compound as described above is used as a tracer; ii) administering the potential anti-cancer treatment to the subject; and iii) repeating step i) to determine the effectiveness of the potential anti-cancer treatment.

In certain embodiments, the present invention provides a method of determining the presence of a cell having up-regulated TSase enzyme activity comprising administering the compound as described above to the cell, and imaging the cell by means of positron emission tomography (PET), wherein the compound is employed as a tracer.

The present compounds are also useful for ex vivo and in vitro methods of imaging.

The present invention provides a method for tissue imaging comprising the steps of contacting a tissue that contains folate receptors with a radiotracer as described above, and detecting the radiotracer. In certain embodiments, the radiotracer detected in vitro. In certain embodiments, the radiotracer detected ex vivo.

The present invention provides a use of a compound as described above for imaging cancer.

The present invention provides a molecule or conjugate as described above for use in the imaging of cancer.

### BRIEF DESCRIPTION OF DRAWINGS

FIG. 1. Schematic representation of designed trapping ¹¹C-radiotracer in a typical cancerous cell. ¹¹C-MTHF is sequestered and uptaken by FR-α and then the radionuclide (C-11) is transferred to dTMP by TSase and thus retained in the cell by metabolic conversion of dUMP to dTMP.

FIG. 2. Stability profile of MTHF in culture medium. MTHF in medium after specific time point was used to convert dUMP to dTMP catalyzed by ecTSase, and the amount of dTMP was analyzed by HPLC.

FIG. 3. Equilibrium between THF and formaldehyde and the irreversible oxidative cleavage of THF. Other oxidative paths (e.g., to DHF or hydroxylation of the C4) are also likely to contribute to irreversible decomposition of MTHF.

FIG. 4. Incorporation of ¹⁴C-MTHF by normal and cancerous cells derived from the breast and colon tissues. The uptake is in units of counts per minute (CPM) per million cells. The cells in log phase were placed in labeling medium containing 20 μM of MTHF and the amount of the tracer retained in the cells were measured by LSC and normalized with total cell numbers and the amount of radiotracer added in the medium. Measurements were conducted in triplicates or duplicates and the variation was less than 5%.

FIG. 5. Percentage release of radioactivity after the cells were placed in fresh growth media and subject to successive post-incubation washes. Culture medium was changed after every 30 min and analyzed for radioactive release by LSC. Measurements were conducted in triplicates or duplicates and the variation was less than 5%.

FIG. 6. Retention of radioactivity in cells. The cells were successively washed by placing in fresh medium for 30 min/wash. After 2 h, the cells were harvested and radiotracer retained by cells was measure in LSC after lysing the cells. Measurements were conducted in triplicates or duplicates and the variation was less than 5%.

FIGS. 7A-7B. Western blot analysis of (A) folate receptors, and (B) TSase in cell pelts. Measurements were conducted in triplicates or duplicates and the variation was less than 5%.

FIG. 8. The conversion of¹⁴CH₂O to ¹⁴C-MTHF in DMF at 70° C.

FIG. 9. The schematic diagram of the module developed for the synthesis of ¹¹C-MTHF.

FIG. 10. HPLC chromatogram of [¹¹C]-MTHF. The top panel is the UV chromatogram, and the bottom panel is the radiogram.

### DETAILED DESCRIPTION OF THE INVENTION

Labelled Compounds

The invention encompasses radioactive tracer forms of the compounds, which have radioactive atoms bonded to them to facilitate in vivo and in vitro imaging detection and quantification methods. The chemical attachment of positron emitting atoms, and similar radionuclides, to certain forms of the agents and at select regiochemical locations affords radioligands (tracers).

In the context of certain embodiments of the present invention, a labelled compound has one or more atoms replaced by an atom having an atomic mass or mass number different from the atomic mass or mass number usually found in nature. Such labelling allows easy quantitative detection of the compound in question. In certain embodiments, the present invention provides a compound of [11-¹¹C]—(R)—N⁵,N¹⁰-methylene-5,6,7,8-tetrahydrofolate (¹¹C-MTHF). In certain embodiments, the present invention provides a compound of [11-¹⁴C]—(R)—N⁵,N¹⁰-methylene-5,6,7,8-tetrahydrofolate (¹⁴C-MTHF). In certain embodiments, the present invention provides a compound of [¹¹C]-leucovorin (folinic acid). In certain embodiments, the present invention provides a compound of 5-[¹¹C]-methyl-THF.

The labelled compounds of the invention may be useful as diagnostic tools, radio tracers, or monitoring agents in various diagnostic methods, and for in vivo and in vitro receptor imaging.

The labelled isomer of the invention preferably contains at least one radionuclide as a label. Positron emitting radionuclides are all candidates for usage. In the context of this invention the radionuclide is preferably selected from ¹¹C and ¹⁴C.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

Cancer Cell Imaging

The radioligands are imaging tracers that detect and quantify cancer/tumor cell densities within live and post-mortem tissues. The term tissue means a part of a subject's or patient's body. The tissue imaging methods encompass positron emission tomography (PET), single photon emission computed tomography (SPECT) and autoradiography. For live tissue imaging, preferably the radiotracers of the invention can be administered to subjects in an amount suitable for in vivo imaging thereof, and to locate, diagnose, identify, evaluate, detect and/or quantify cancer cells. Generally, a unit dosage comprising a radiotracer of the invention may vary depending on subject or patient considerations. Such considerations include for example, age, condition, sex, extent of disease, contraindications, or concomitant therapies.

The MTHF, folinic acid and methyl-THF derivatives of the present invention are useful as diagnostic tools or monitoring agents in various diagnostic methods, and in particular for in vivo receptor imaging.

In another aspect of the invention, a method for the non-invasive determination of the distribution of a tracer compound inside a whole, intact living animal or human body using a physical detection method is provided. According to this method a tracer compound is a compound of the invention, or any of its enantiomers or any mixture thereof, or a pharmaceutically acceptable salt thereof, in labelled or unlabelled form.

In certain embodiments, the physical detection method is selected from Positron Emission Tomography (PET), Single Photon Emission Computed Tomography (SPECT), Magnetic Resonance Spectroscopy (MRS), Magnetic Resonance Imaging (MRI), Computed Axial Tomography (CAT), Computed Tomography (CT), Functional Magnetic Resonance Imaging (fMRI), or combinations thereof.

Before conducting the method of the present invention, a diagnostically effective amount of a labelled or unlabelled compound of the invention is administered, e.g., intravenously, to a living body. The diagnostically effective amount of the labelled or unlabelled compound of the invention to be administered before conducting the in vivo method for the present invention is within a range of from 0.1 ng to 100 mg per kg body weight, preferably within a range of from 1 ng to 10 mg per kg body weight.

Pharmaceutical Compositions

The imaging compounds of the present invention can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508). Useful dosages of the imaging compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949.

The compounds of the invention may also be administered to a subject or patient with other therapeutic agents that may be useful in the treatment of cancer. A method is provided for administering an effective amount of one or more compounds of the invention to a subject suffering from or believed to be at risk of suffering from cancer. The method also comprises administering either sequentially or in combination with one or more compounds of the invention a conventional therapeutic measure protocol or agent that can potentially be effective for the treatment or prophylaxis of a cancer.

Administration of a compound or radiotracer of the invention to a subject may be local or systemic and accomplished orally, intradermally, intramuscularly, subcutaneously, intravenously, intraaterially or intrathecally (by spinal fluid); or via powders, ointments, drops or as a buccal or nasal spray. A typical composition for administration can comprise a pharmaceutically acceptable carrier for the compound or radiotracer of the invention. Pharmaceutically acceptable carrier include, without limitation, aqueous solutions, non-toxic excipients comprising salts, preservative or buffers, amongst others known within the art.

In one embodiment, a composition can also comprise a pharmaceutically acceptable carrier and compound or radiotracer of the invention. A composition of the invention can be administered to a subject by conventional techniques including, without limitation, by a bolus injection.

The invention will now be illustrated by the following non-limiting Examples.

### EXAMPLE 1

### In Vitro Uptake of N⁵,N¹⁰-Methylenetetrahydrofolate by Normal and Cancerous Cells: Development of Novel Positron Emission Tomography Tracer

Development of tumor specific probes for imaging by positron emission tomography (PET) has broad implications in clinical oncology such as diagnosis, staging, and monitoring therapeutic responses in patients, as well as in bio-medical research. Thymidylate synthase (TSase)-based de novo biosynthesis of DNA is an important target for drug development. Increased DNA replication in proliferating cancer cells requires TSase activity, which catalyzes the reductive methylation of 2′-deoxyuridine 5-monophosphate (dUMP) to 2′-deoyxythymidine 5-monophosphate (dTMP) by using (R)—N⁵,N¹⁰-methylene-5,6,7,8-tetrahydrofolate (MTHF) as a co-factor. In principle, radiolabeled-MTHF can be used as a substrate for this reaction to identify rapidly dividing cells. In this Example, actively growing (log phase) breast cancer (MCF7, MDA-MB-231, hTERT-HME1), normal breast cells (HMEC, MCF10A), colon cancer (HT29), and normal colon (FHC) cells were incubated with ¹⁴C-MTHF in culture medium from 30 min to 2 h and uptake of radiotracer was measured. Cancerous cell lines incorporated significantly more radioactivity than their normal counterparts. The uptake of radioactively labeled MTHF depended upon a combination of cell doubling time, folate receptor status, S-phase percentage as well as TSase and folate receptors expression in the cells. These findings suggest that the recently synthesized ¹¹C-MTHF may serve as a new PET tracer for cancer imaging.

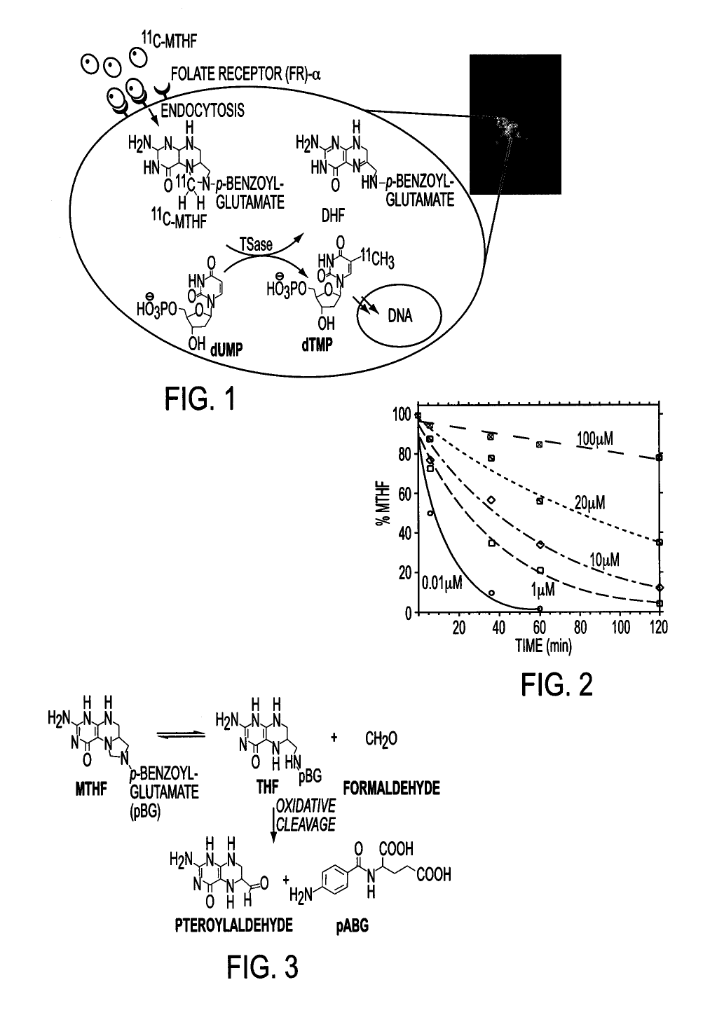

Thymidylate synthase (TSase, E.C. 2.1.1.45) plays a central role in the de novo biosynthesis of the DNA base thymine in humans. It is over expressed in many cancers, making it a good target for a diagnostic probe to detect rapidly growing cells. It catalyzes the reductive methylation of 2′-deoxyuridine-5′-monophosphate (dUMP) to 2′-deoxythymidine-5′-monophosphate (dTMP), where the methyl group is provided by the methylene from its cofactor, (R)—N⁵,N¹⁰-methylene-5,6,7,8-tetrahydrofolate (MTHF). We incorporated a radionuclide (C-11 or C-14) into the methylene carbon of MTHF so that the radiolabelled methylene would be incorporated into dTMP and its down-stream products toward DNA. When selectively incorporated into cancer cells, the ¹¹C-MTHF is used as a PET imaging probe (FIG. 1). The synthesis of ¹¹C-MTHF is presented in Example 2 below, but due to the short half-life of C-11 radionuclide (20.4 min), the inventors used the chemically equivalent ¹⁴C-MTHF for the in vitro studies with cell culture presented here. The results show that the radioactivity is selectively incorporated in cancer cells and that MTHF can be used as a cancer detection probe.

Experimental Procedures

Materials—¹⁴CH₂O (50 mCi/mmol, 1-3% aqueous solution) was purchased from Moravek Biochemicals (Brea, Calif.). Folic acid, TES, dUMP, and dTMP were purchased from Sigma. Recombinant E. coli TSase (ecTSase) was expressed and purified from E. coli culture, according the procedures reported by Changchien et al. (Changchien, L. M., Garibian, A., Frasca, V., Lobo, A., Maley, G. F., and Maley, F. (2000) Protein Expr Purif 19, 265-270). DHF, THF, MTHF and ¹⁴C-MTHF were prepared from folic acid as reported before (Agrawal, N., Mihai, C., and Kohen, A. (2004) Anal Biochem 328, 44-50).

HPLC analysis—Analytical HPLC was conducted on a System Gold® 126 solvent module connected with a System Gold® 168 detector (Beckman Coulter, Calif.) for UV monitoring and a flow scintillation analyzer 500TR series (Packard Bioscience, Mass.) for C-14 radionuclide monitoring.

Stability of MTHF in culture medium—Folate-free RPMI 1640 (Invitrogen), fortified with 10% Fetal bovine serum (FBS, Sigma, qualified) (1 mL) in an eppendorf tube (1.5 mL) was spiked with MTHF (10 nM-100 μM) containing ˜0.05 μCi (˜1×10⁵ DPM) of ¹⁴C-MTHF as a radiotracer, and incubated under normal culture conditions (see below) for 0, 5, 30, 60, and 120 min. At a desired time point, an aliquot of the culture medium (100 μL) was removed and mixed with 100 μl of TES buffer containing 1 mM dUMP and 10 U of ecTSase to convert the radiotracer from MTHF to the more stable dTMP (Hong, B., Maley, F., and Kohen, A. (2007) Biochemistry 46, 14188-14197; Agrawal, N., Hong, B., Mihai, C., and Kohen, A. (2004) Biochemistry 43, 1998-2006; Agrawal, N., Mihai, C., and Kohen, A. (2004) Anal Biochem 328, 44-50). The mixture was incubated at 30° C. for 15 min and then filtered through 10K MWCO filter to remove protein. A 100 μl filtrate was analyzed by HPLC to determine the amount of dTMP formed from MTHF. A zorbax column (4.6 mm×15 cm) was eluted with a gradient of 2% to 5% methanol in 100 mM potassium phosphate, pH 6 over 2 min. The isocratic elution at this composition was conducted for 3 min and then changed linearly to 50% methanol in next 5 min. The system was held at this composition for next 5 min, and then increased up to 95% methanol to clean the column. Under these conditions, dUMP was eluted at 3.2 min, dTMP eluted at 5.8 min and MTHF/DHF eluted at 16 min. Identification and quantitation of dTMP was accomplished by comparing the retention time and area under peak of the standard solutions of dTMP.

Cell culture—Breast cancer cell lines (MCF7, hTERT-HME1, MDA-MB-231), normal breast cell lines (HMEC, MCF10A), and colon cancer cell line (HT29) were kindly provided by Douglas Spitz, University of Iowa. Primary human colon (FHC) cells were purchased from ATCC. All cell cultures, but HMEC, were conducted in an incubator maintained at 37° C., 5% CO₂/95% air atmosphere. The HMEC cells were maintained in a low oxygen (4%) incubator at 37° C., 5% CO₂. All cell cultures except HMEC were split when 70-80% confluent. MCF10A, hTERT-HME1 cells—the cells were grown in DMEM/F12 medium (Invitrogen) supplemented with 10% heat inactivated FBS, 1×MEGS (containing bovine pituitary extract, bovine insulin, hydrocortisone and recombinant human epidermal growth factor), 50 IU/ml penicillin, and 50 μg/ml streptomycin. The culture was replenished with medium after every 72 h. The cells at 70-80% confluency were used for uptake experiment. MCF7, MDA-MB231, HT-29 cells were grown in RPMI-1640 medium supplemented with 10% heat inactivated FBS, 50 IU/ml penicillin, and 50 μg/ml streptomycin. The culture was replenished with medium after every 72 h. The cells at 70-80% confluency were used for uptake experiment. FHC cells were maintained in DMEM/F12 medium (Invitrogen), supplemented with 10% heat inactivated FBS, 10 ng/ml cholera toxin (Sigma), 5 μg/ml insulin (Sigma), 5 μg/ml transferring (Sigma), 100 ng/ml hydrocortisone (Sigma), 50 IU/ml penicillin, and 50 μg/ml streptomycin. The medium was replaced after every 72 h. The cells at ˜100% confluency were used for uptake experiment. HMEC cells were grown in a basal medium (MEBM®, Lonza), which was supplemented with SingleQuots® (MEGM® BulletKit®, Lonza). Cells were passed at 1×10⁵/ml. The medium was replaced after every 48 h. To mimic the slow growth in normal mammary gland, HMEC cells were grown up to 100% confluence, maintained in the same medium for 24 h and then used for uptake experiments.

Uptake of ¹⁴C MTHF—Cancerous cells were grown to 70-80% confluency. Before the start of uptake experiment, the normal medium was replaced with folate-free RPMI 1640 medium for 30 min. Labeling media constituted folate-free RPMI 1640, supplemented with 5% dialyzed FBS, 1× glutamine and 50 IU/ml penicillin, and 50 μg/ml streptomycin. A volume of 30 mL pre-warmed uptake medium was spiked with MTHF (20 μM final concentration containing ˜0.5 μCi ¹⁴C-MTHF. The old medium from the cells was aspirated and 3 mL of labeling medium was added per 10 cm² plate. The cells were incubated at 37° C. for 30, 60, 90, 120, and 180 min. After treatment, the medium was removed and cells were washed twice with cold PBS buffer. The cells were detached with 0.25% trypsin-EDTA solution, and counted using a Coulter Counter (Beckman Coulter, Calif.). The cell pellet was lysed in 0.5 mL of 0.5% SDS, mixed with scintillation fluid and counted in scintillation counter for 5 min using the ¹⁴C window.

Washout of label from cells—After labeling cells grown in four plates as described before, one culture plate was processed and total incorporation was measure by liquid scintillation counter (LSC). This uptake was regarded as 100%. From the remaining three culture plates, medium was removed, cells were washed twice with cold PBS and then normal growth medium (5 mL) was added at 37° for 30 min. The radiotracer released in the medium after 30 min was counted by LSC in triplicate, and cells were replenished with fresh 5 mL medium for next 30 min washing. The release of radiotracer was followed up to 120 min. Measurement of cell growth rate-The cells were seeded at a density of 4×10⁴ cells/cm² in each of four 6-cm (20 cm²) plates containing 5 mL medium and placed in incubator maintained at the conditions described above. Once the cells were in log-phase (48 h post plating), two plates were processed to count the total cells. The average number of cells from these plates was regarded as pl. After next 24 h, the remaining two plates were processed, and the total cells were counted again (p2). The doubling time was then calculated by using formula

Doubling time (h)=24'ln2/ln(p2/p1)

Western blot analysis of FR-α and TSase enzyme—Culture medium of the Cells in log phase was aspirated and washed twice with cold PBS. The cells were then harvested on ice with 200 μL of cold lysis buffer containing 1% Triton X100, 0.5% sodium deoxycholate, 0.1% SDS, 1 mM sodium orthovanadate and 1 tablet of complete mini EDTA-free cocktail. The extract was incubated on ice for 20 min, passed through a 25 gauge needle and finally centrifuged (Eppendorf Centrifuge 5403) at 15,000 g for 20 min at 4° C. The supernatant was removed in 1.5 mL tube, and protein concentrations were measured by BCA protein estimation kit. A protein sample of 50 pg was loaded and electrophoresed in 10% SDS-PAGE gels. Size standards from 10 kDa-250 kDa (BioRad Kaleidoscope) were included. Polypeptides were electrotransferred to nitrocellulose membrane. The membranes were blocked with 5% non-fat milk in TBST (3% BSA, 0.2% Tween 20 in phosphate buffered saline (PBS)). The membranes were then exposed to anti-human folate receptors (Santa Cruz Biotechnology) or anti-human TSase (Santa Cruz Biotechnology), and then treated with antibodies labeled with Alexafluor-680 or Alexafluor-800. The membranes were then blotted on a Li—COR Odyssey scanner.

Cell cycle analysis—Cells in log phase at the same confluency used for uptake experiments or growth inhibited cells by aphidicolin treatment were harvested using trypsin and washed twice with cold PBS. The cells were suspended as 1×10⁶/ml density in PBS and fixed with 70% ethanol at −20° C. The DNA content of the cells was stained with 1 mL of staining buffer containing propidium iodide (5 μg/ml), RNAse A (1 mg/ml), glucose (1 mg/ml) in PBS. The stained cells were filtered through 70 μm nylon strainer and then analyzed for DNA content on FASCan flow-cytometer (Becton Dickinson) using Flo-Jo software.

Results and Discussion

Stability of MTHF in culture medium—The stability of MTHF might limit its pharmaceutical use. In most part, that stability is determined by the hydrolysis of the methylene as formaldehyde and the sensitivity of the product THF to oxygen. The inventors therefore measured the stability of MTHF in culture medium. Concentrations of MTHF ranging from 10 nM to 100 μM, containing a fixed amount of radiotracer (C-14) were incubated in culture medium, under the conditions used for cell incorporation experiments. MTHF remaining in the medium at different time points was measured by quantitatively converting dUMP +¹⁴C-MTHF to ¹⁴C-dTMP by the ecTSase-catalyzed reaction. The amount of ¹⁴C-dTMP formed was then analyzed by HPLC connected with radioactive flow detector (FIG. 2). Data were fitted to a single exponential decay. The analytical assay in which ¹⁴C-MTHF is quantitatively converted to ¹⁴C-dTMP was developed, tested, and used in many of our kinetic isotope effect measurements while studying TSase. In this case the range of 10 nM to 100 μM MTHF was measured with 10 Units of ecTSase for 15 min and we found that all the [¹⁴C]-MTHF radioactivity has been converted to [7-¹⁴C]dTMP (>99.9%). Since the lower range was not found stable, we did not test the amounts less than 10 nM.

From FIG. 2, it is clear that the decomposition of MTHF does not follow first order kinetics and its rate is concentration dependent. The rate of decomposition k_{dec}=7.27×10⁻²/min at 10 nM MTHF and this rate decreases to k_{dec}=1.94×10⁻³/min with increasing concentration of MTHF to 100 μM. The concentration-dependent stability of MTHF up to 330 min at 100 μM is sufficient for labeling cell cultures. Although the mechanism of concentration dependent MTHF stability is not clear, it may be related to equilibrium with formaldehyde in the solution. Since MTHF in aqueous solution hydrolyzes to formaldehyde and THF (FIG. 3), and the reverse reaction is in competition with irreversible C⁹-N¹⁰ oxidative cleavage of THF into pteridine-6-aldehyde and p-aminobenzoyl-L-glutamate (pABG), as well as oxidation of THF to DHF and other oxidation derivatives. At very low concentrations of MTHF, the released formaldehyde diffuses into solution leaving the THF unprotected and oxidative cleavage reactions quickly shift the equilibrium toward further hydrolysis.

To obtain sufficient MTHF for labeling with a sufficiently long half-life, and yet not risk overdose of folate in the medium, we selected a concentration of 20 μM to be a suitable dose of MTHF with a reasonable stability (half-life 78 min). This choice is made so the time scale of the in vitro experiments described below is relevant to the short living ¹¹C to be used in vivo.

Uptake of MTHF by normal and cancerous cell lines. It was proposed that cancer cell lines may be inherently more capable of incorporating exogenous MTHF than their normal cell counterparts. To test this proposal, different cell lines derived from breast (MCF7, MDA-MB231, hTERT-HME1) and colon cancers (HT-29) as well as their normal counterparts (HMEC, MCF10A for breast and FHC for colon) were grown in their respective growth media and maintained in log phase. Before the start of uptake experiment the normal growth medium was removed and replaced with folate-free medium for 30 min to remove any folate attached to the receptors on membrane and let the cell slightly “starve” of folates. It should be noted that this short folate starvation does not significantly deplete intracellular poly-glutaminated folate reserves, which may last for several days under these conditions (data not shown). Cells were incubated with 20 μM MTHF containing 0.05 μCi of C-14 tracer for different time periods (0.5, 1, 1.5, 2 h). The radiotracer taken up by the cells was determined by LSC and normalized with the total cell numbers and initial dose of radioactivity as calculated by formula:

$$Normalized uptake = \frac{Cpm recorded in cells}{{Cpm}_{added} \times Total {cell}_{i n millions}}$$

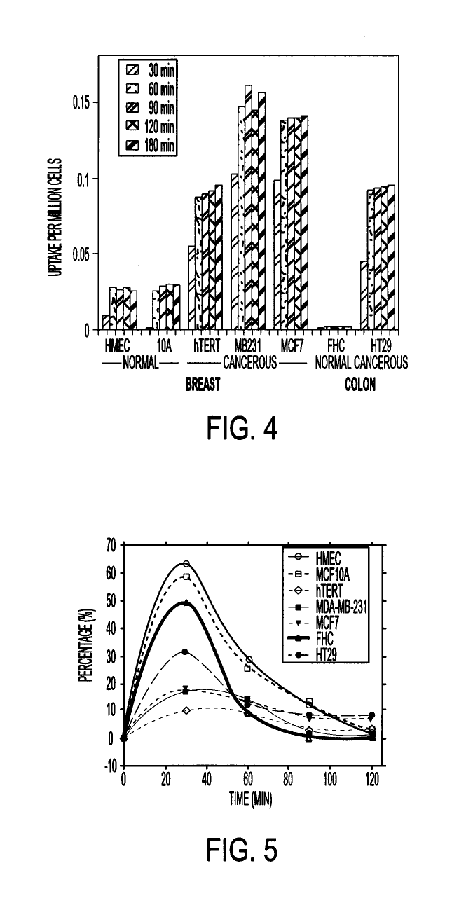

As shown in the FIG. 4, cancerous cells lines (HEMC-hTERT; MCF7 and MDA-MB231) took up more than 3 fold the amount of label incorporated into non-cancerous control cell lines (HMEC, MCF10A). In case of colon cancer cell line (HT29) the uptake was at least 30 times more predominant than in normal FHC cells, for which uptake was not higher than background. Interestingly the spontaneous immortalized “normal” cell line (MCF 10A) also showed less uptake as compared with the cancerous cells.

MTHF can be both taken up by the cell and, to an extent, lost from the internal pool to the cell culture media. The time taken to reach steady state reflects the relative rates of these two processes. Uptake of label by cancerous cell lines reached a maximum level after 60 min. Incubation for a time longer than 60 min did not change the amount of label incorporated into the cell, indicating that the intracellular pool was saturated and at steady state.

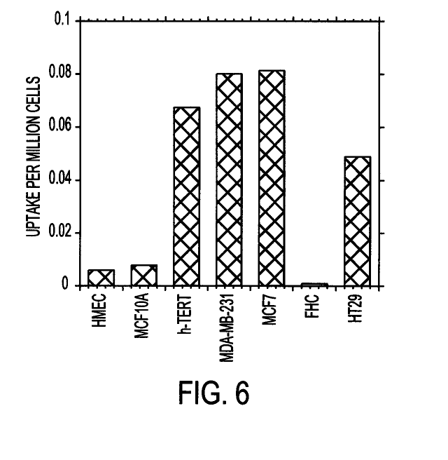

To examine the release of radioactivity from these cells, the ¹⁴C-MTHF-loaded cells (after 2 hours uptake) were placed in fresh complete medium containing an excess of unlabeled folate at 37° C. The medium was replaced after every 30 min and the radiotracer released in the medium was measured by LSC. As shown in FIG. 5, highest release of radiotracer was observed in case of normal cells, where >50% tracers was released in first 30 min incubation. In case of cancerous cell lines, the release of the radiotracer was not more than 20%. After 30 min, the release of the radioactivity in the medium was slow and reached to the background value after two successive washings. After washing, the label retained in each cell line was determined and plotted in FIG. 6. As the plot shows, the breast cancer (MDA-MB231, MCF7, hTERT) and colon cancer (HT29) cell lines retained the highest amount of tracer, whereas their non-cancer counterparts retained a much lower, near-background amount of label. This suggested that the label associated with cancer cell lines was significantly more likely to be retained than that in the non-cancer cell lines.

In order to better understand why cancer cell lines take up and retain more MTHF, three cellular parameters were examined and correlated with the amount of radiotracer retained by the cells. These included cell doubling time, plasma membrane folate receptor (alpha subunit), and TSase enzyme protein level.

Doubling time vs. ¹⁴C-MTHF uptake. A count of 40,000/cm² cells from each line was grown in 6-cm (20 cm²) plates in their recommended media. As the doubling time is more reliably measured in log phase, the cells were re-counted 48h after splitting to establish the population (p1), and again at 72 h to establish population (p2). Primary cells from breast (HMEC) and colon (FHC) were the slowest growing cells with a doubling time of 124.2±5.5 hours and 76.2±8.5 hours, respectively. Spontaneously immortalized breast MCF10A cells doubled in 24 h. On the other hand the cancerous cell lines, once in log phase, had doubling times ranging from 20 h to 31 h (Table 1). A negative correlation (r=−0.63) was observed between the doubling time of cells and MTHF uptake. This observation is in accordance with the fact that the cells having short doubling time need enhanced synthesis of thymine base and thus indicated enhanced MTHF uptake. Since the intracellular thymine base pool can be replenished by two distinct and unique pathways, namely salvage pathway and de novo synthesis, the information of cell doubling time is not sufficient to show the dependence of MTHF uptake with cell proliferation rate. Therefore, we examined the FR status and TSase protein expression level in the treated cells.

TABLE 1
Doubling time, % age of S- + G2M phase and uptake of MTHF in normal and
growth inhibited cells

| | | Normal growing cells | | aphidicolin-arrested cells | |
|---|---|---|---|---|---|
| | | Proliferation index | MTHF uptake | Proliferation index | MTHF uptake |
| Cell type | Doubling time, h | (S + G2M), % | (×10⁻³/10⁶ cells) | (S + G2M), % | (×10⁻³/10⁶ cells) |
| HMEC | 124.2 ± 5.5 | 11 | 5.98 | 4 | 1.23 |
| MCF-10A | 24.1 ± 1.5 | 12 | 7.79 | 3 | 2.34 |
| hTERT- | 20.4 ± 2.5 | 23 | 67.3 | 6 | 5.74 |
| HME1 | | | | | |
| MDA- | 27.8 ± 1.3 | 25 | 81.3 | 6 | 3.45 |
| MB231 | | | | | |
| MCF-7 | 31.0 ± 1.6 | 24 | 80.0 | 5 | 4.92 |
| FHC | 76.2 ± 8.5 | 9 | 0.94 | 3 | 0.54 |
| HT-29 | 22.4 ± 2.5 | 19 | 48.9 | 6 | 3.82 |

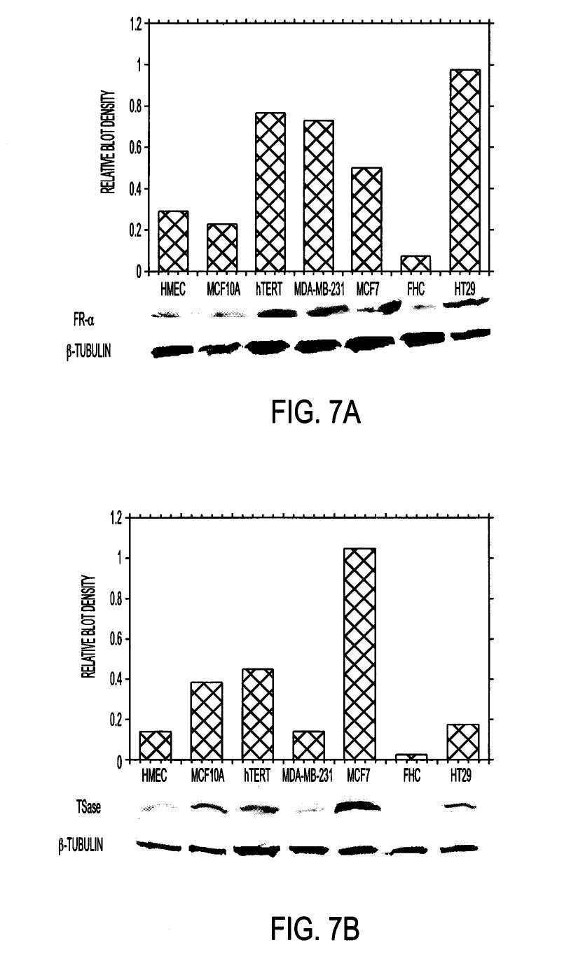

Western blot analyses of folate receptor and TSase protein Expression. Folic acid and reduced folates are transported across the plasma membrane via the Folate Receptor (FR). A number of human cancers are reported to overexpress FR-α, and cytosolic TSase protein (34-38). To observe the dependence of uptake of ¹⁴C-MTHF on FR-α expression, a western blot for FR-α was performed on all cell lines (FIG. 7).

All the cell lines used in this study showed the presence of FR-a, however level of protein expression was different for each cell line (FIG. 7A). Cancer cell lines had approximately twice as much FR-a protein expression as that observed for non-cancerous cell lines. Density of the blots due to FR-a was determined and normalized with that of β-tubulin and then plotted for each cell line (7A top panel). A good correlation (r=0.74 between the level of FR-α protein expressed on the membrane and the level of MTHF uptake suggested that at least part of the increased incorporation of label by cancer cell lines could be attributed to FR-α expression.

Since MTHF is a co-factor of TSase that converts dUMP to dTMP, induction of de novo pathway for DNA synthesis may render the expression of this enzyme, which could be another important parameter for the retention of ¹⁴C-MTHF radioactivity in cells. Therefore, we sought to find a correlation between MTHF tracer uptake in the cells and the expression of TSase enzyme level. The levels of TSase enzyme were determined by western blot analyses (FIG. 7B). Apparently, TSase tended to be more highly expressed in cancer cell lines. Cancerous MCF7 cells, for example, expressed far more than normal breast (HMEC) and colon cells. However, only a fair correlation between TSase and label incorporation was observed (r=0.54). Interestingly, when taken together, the total effects of expression of FR-α and TSase protein correlated strongly (r=0.86) with the MTHF uptake. This indicated that for a predominant and specific MTHF uptake, over expression of both proteins FR-α, on plasma membrane and TSase in cytoplasm is required. The cancerous cell lines exhibiting over expression of both proteins than their normal counterparts tended to incorporate more MTHF.

Cell cycle vs. MTHF uptake. Biosynthesis of DNA and its precursor bases increases during S-phase of the cell cycle. Therefore, we sought to explore the correlation between the MTHF uptake and proliferation index (percentage of cells in S+G2M phase) exhibited by each cell line. Unsynchronized cells, as used for labeling, were alcohol-fixed and the DNA stained with propidium iodide (PI). DNA content can be used as a proxy for determining cell cycle stage. The DNA content was then analyzed by FACSCAN flow cytometer and data were processed by using Facscaliber software. As shown in the Table 1, the cells grown in normal growth medium showed high proliferation index, which is highly correlated (r=0.99) with the uptake levels exhibited by these cells. Alternatively, when the cells were treated with aphidicolin, an inhibitor of DNA polymerase-α, the proliferation index is reduced up to 4-fold. Labeling of cells arrested in S-phased by aphidicolin showed a marked reduction in the tracer retained in these cells (table 1). These results suggest that cells passing through S phase take up the most MTHF, linking the radioactivity uptake to enhanced cell proliferation. In another experiment, we treated cells with a chemotherapeutic agent (methotrexate) for 24 h and then incubated with ¹⁴C-MTHF for 1 h and observed a 90-95% decline in uptake relative to cells not treated with methotrexate.

In conclusion, the findings presented here demonstrated that radioactivity from labeled MTHF is incorporated and retained more by cancerous cell lines than by non-cancerous cell lines. This labeling is primarily dependent upon the higher expression of Folate receptor in the cancerous cell lines and, to a lesser extent, by the higher level of TSase. The cells exhibiting higher proliferating index (portion of cells in S+G2M phase) uptake more MTHF than the non-proliferating cells. Together, these results show that methylene-labeled MTHF can be used as a specific and unique probe for the labeling of cancer cells.

### EXAMPLE 2

### Preparation of [¹¹C]-labeled precursor for thymidylate

Positron emission tomography (PET) is an emerging clinical tool for cancer diagnosis and treatment. Development of PET probes that can selectively target tumors is essential for the success of this technology. In 2008, the preparation of the positron emitting reagent ¹¹C-formaldehyde was reported by Hooker and co-workers (J. M. Hooker, M. Schonberger, H. Schieferstein, J. S. Fowler, Angew Chem Int Ed Engl 2008, 47, 5989-5992). Herein we report the use of that reagent in the synthesis of [11-¹¹C]—(R)—N⁵ ,N¹⁰-methylene-5,6,7,8-tetrahydrofolate (¹¹C-MTHF), which is a co-factor for the enzyme thymidylate synthase (TSase, E.C. 2.1.1.45) and is transported into cells via folate receptors (FRs). Up-regulated TSase activity and over-expressed FRs in tumors are targeted for the uptake and retention of the ¹¹C in the DNA base thymine. A fast chemo-enzymatic synthetic path was used to prepare the potential radiotracer ¹¹C-MTHF. The synthesis and purification were conducted remotely in a simple synthetic module in ˜5 min. The preparation of ¹¹C-MTHF leads to new non-invasive methods of cancer imaging, which help in the clinical diagnosis of cancers as well as guide of treatments.

Non-invasive imaging technologies are widely used clinical tools for the diagnosis, staging, restaging, and monitoring therapeutic response of cancer. Different technologies have been developed to image the structure and function of systems, such as positron emission tomography (PET), magnetic resonance imaging (MRI), X-ray computed tomography (CT). Among those, PET is unique to render metabolic, biochemical, and functional activity in vivo, contrary to CT and MRI, which primarily provide images of organ anatomy. Since morphological response to chemotherapy or radiation therapy lags behind the course of the treatment, PET is more effective in assessing therapeutic response.

PET is a non-invasive imaging technology developed to use tracers labeled with short lived positron emitting radioisotopes as molecular probes to image and measure biochemical processes in vivo. The most extensively used PET probe is ¹⁸F-fluorodeoxyglucose (¹⁸F-FDG), which probes glucose metabolism as it is transported into the tissue by glucose transporters and then phosphorylated by hexokinase. The 6-phosphate is not a substrate for the next enzyme in the glucose metabolic pathway and on the time scale of fluorine-18 the tracer accumulates in the tissue as the FDG 6-phosphate. It is exquisitely sensitive to the hexokinase reaction but this reaction is not limited to tumors. A newer radiotracer 3′-deoxy-3′-fluorothymidine (¹⁸F-FLT) is coming into use as a marker of thymidylate kinase activity, which is inflated in some rapidly proliferating tumors. However the low yield on the synthesis of FLT has limited its commercial distribution. In principle, ¹⁸F-labeled metabolites are never a natural substrate, i.e., these are chemically modified and commonly have an inhibitory role along the metabolic pathway. Metabolites labeled with ¹¹C, however, are natural and do not cause a perturbation of the metabolic pathway under study (¹¹C is chemically almost identical to the common isotope ¹²C, and its isotope effects are negligible).

This Example describes the synthesis of a new radiotracer using the positron emitting radionuclide carbon-11, which is designed to target the thyA-coded TSase. TSase catalyzes the only de novo biosynthesis of the DNA building block T, i.e., 2′-deoxythymidine-5′-monophosphate (dTMP) in human, which is found to be over-expressed in tumor tissue. Consequently, TSase is a target for fluoropyrimidine-based drugs such as 5-fluorororuracil. Furthermore, intrinsic tumor TSase values are also thought to be of importance in predicting the response to chemotherapy and survival in patients. Patients with low tumor TSase expression have a significantly better outcome than those with high TSase expression.

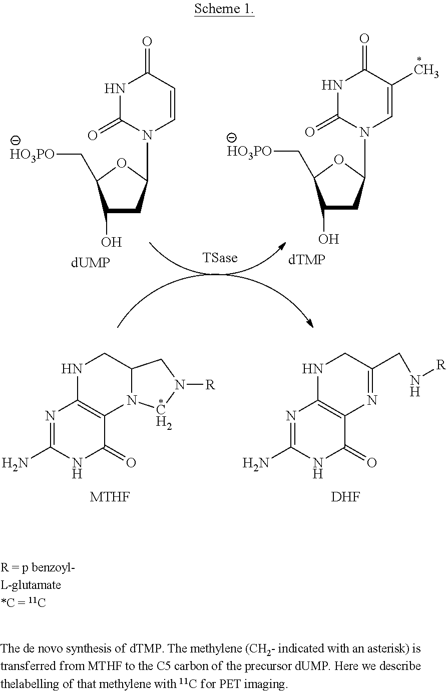

TSase catalyzes the reductive methylation of 2′-deoxyuridine-5′-monophosphate (dUMP) to dTMP. The cofactor (R)—N⁵,N¹⁰-methylene-5,6,7,8-tetrahydrofolate (MTHF) first donates a methylene group to form (S)-5,6,7,8-tetrahydrofolate (THF), then a hydride to produce dTMP and 7,8-dyhydrofolate (DHF, Scheme 1). Its mechanism and multiple step chemical conversions have been carefully examined, in part by the use of [¹⁴C]-MTHF (B. Hong, F. Maley, A. Kohen, Biochemistry 2007, 46, 14188-14197; N. Agrawal, B. Hong, C. Mihai, A. Kohen, Biochemistry 2004, 43, 1998-2006; Z. Wang, A. Kohen, J Am Chem Soc 2010, 132, 9820-9825). In proliferating cells, de novo synthesis of dTMP by TSase requires a continuous supply of MTHF. Scheme 1 presents the reaction of MTHF with ¹¹C methylene (CH₂) in red. This radioactive methylene is transferred by the TSase catalyzed reaction to the DNA building block dTMP. In order to be uptaken into cells, exogenous MTHF requires specific transporters. FRs are known to transport many derivatives of folic acid that are structurally similar to MTHF, such as methotrexate (MTX), trimethoprime (TMP), leucovorin, and other anitfolates. Importantly, FR-alpha is over-expressed in many types of human cancers. A subset of proliferating cells exhibiting “a double phenotype”, i.e., up-regulated TSase enzyme and over- expressed FR render MTHF as a unique probe for detecting those cells. Folate receptor will allow the probe to enter the cells whereupon TSase activity may trap it as dTMP, or other downstream anionic molecules such as dTTP or DNA, that cannot leave the cells. To examine the potency and up-take mechanism of ¹¹C-MTHF, preliminary in vitro studies compared the up-take of ¹⁴C-MTHF by various human cancer cell lines vs. their normal counterparts. The findings indicated that indeed the cancerous cells were significantly labeled over the normal ones. Furthermore, the fast proliferating cells also over expressed FR and TSase enzyme, which reflected the higher uptake of the radiotracer.

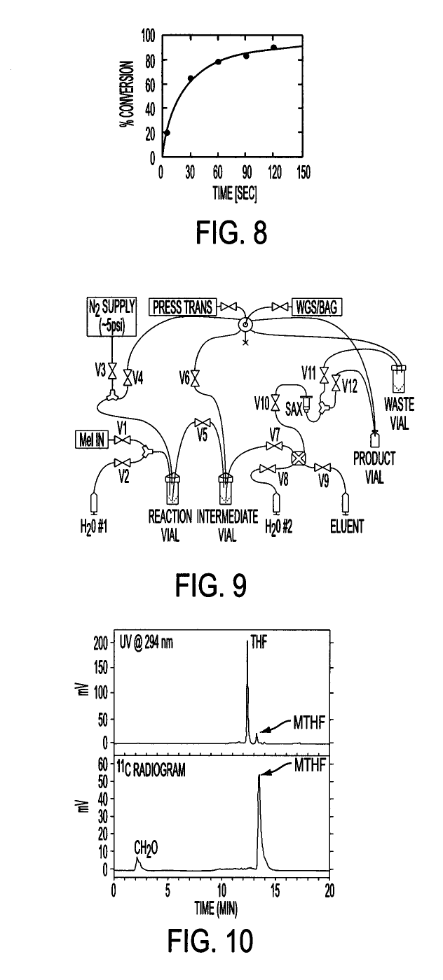

The synthesis of ¹¹C-MTHF begins with a nuclear reaction [¹⁴N(p,α)¹¹C] using 17 MeV protons and a target gas that contains traces of oxygen to produce ¹¹CO₂. ¹¹CO₂ was reduced to ¹¹CH₄ with hydrogen over a nickel catalyst and then converted to ¹¹CH₃I by reaction with iodine at ˜700° C. using PETtrace MeI Microlab (GE Medical Systems, Milwaukee, Wis., USA). Oxidation of ¹¹CH₃I with trimethylamine-N-oxide (TMAO) to ¹¹C-formaldehyde (¹¹CH₂O) was conducted by the fast and high-yield method developed by Hooker and co-workers (J. M. Hooker, M. Schonberger, H. Schieferstein, J. S. Fowler, Angew Chem Int Ed Engl 2008, 47, 5989-5992). As illustrated in Scheme 2, we coupled this reaction with a fast (<1 min) chemo-enzymatic synthesis of MTHF from DHF reported earlier from our laboratory (B. Hong, A. Kohen, Journal of Labelled Compounds & Radiopharmaceuticals 2005, 48, 759-769; N. Agrawal, C. Mihai, A. Kohen, Anal Biochem 2004, 328, 44-50). To assess the kinetics of that process, we replicated the synthetic conditions using ¹⁴CH₃I as starting material, and aliquots of the reaction mixture were removed at different times and analyzed by HPLC equipped with a radioactivity flow detector. We observed that the reaction was about 70% completed within the first minute and up to 80% in the next minute as shown in FIG. 8.

In order to remotely control the synthesis and purification of ¹¹C-MTHF (specific radioactivity of 10-30 Ci/mmol), we developed a synthesis module (FIG. 9). The synthetic and operational procedure is described in detail in the Supplementary Information provided below. In short, the module consisted of a reaction vial to synthesize ¹¹CH₂O and an intermediate vial for mixing THF with the ¹¹C11₂O. Both vials were secured with airtight caps. The reaction vial was placed snugly on a rapid heating-cooling thermoelectric micro-plate. For reagents and carrier gas, the reaction vial cap was supplied with airtight tubing ports, one each for incoming ¹¹CH₃I and H₂O supply, and one for the carrier nitrogen gas. The module was placed inside a lead-lined hot cell with the temperature controller outside.

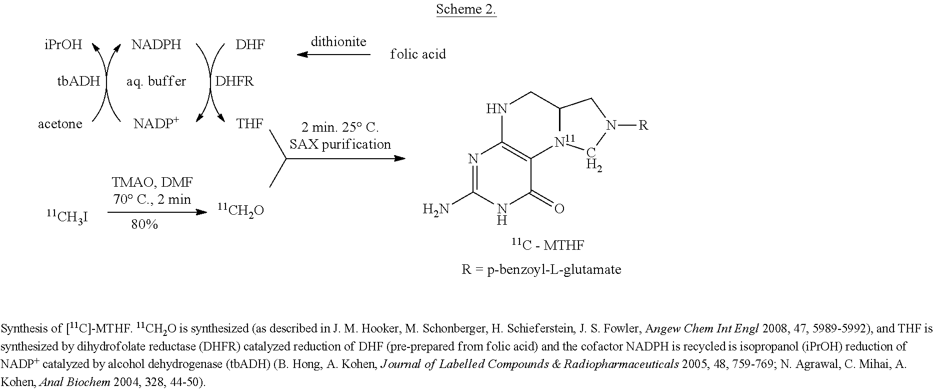

The product collected was then analyzed by HPLC (FIG. 10). The results indicated that most of the impurities (DMF, TMAO etc.) were removed during washing step and radioactive loss from the cartridge, probably as unreacted ¹¹CH₂O, accounts for <10% of the original radioactivity trapped in the reaction vial. The product, ¹¹C-MTHF was cleanly eluted with 100 mM sodium phosphate, pH 7.4 containing 500 mM NaCl and 1% ascorbic acid and HPLC radiogram, which indicated that this product accounts for >95% of the radioactivity with trace amounts of ¹¹CH₂O (FIG. 10). The UV trace indicated that THF (major peak) and MTHF (minor peak) were the only two products. Since THF is a non-toxic natural derivative of folic acid (LD₅₀=10 g/Kg; mouse), and due to the short life of ¹¹C, we chose not to purify this mixture further and use it as is in future in vivo experiments. Additionally, the elution buffer (100 mM sodium phosphate, 500 mM NaCl, pH 7.4) is a physiologically appropriate for in vivo studies.

In summary, an expeditious preparation of [¹¹C]-MTHF, ready in physiological solution, was developed by combing two fast and high yield methods i.e., a fast conversion of cyclotrongenerated ¹¹CO₂ to ¹¹CH₃I, and its conversion to¹¹CH₂O, which is then reacted with chemo-enzymatically prepared THF. The feasibility of its use in PET imaging of cancer was assessed using a longer-lived isotope (¹⁴C) in cell cultures as described in Example 1 above. Those experiments also revealed a contribution of over-expressed FR-a and TSase to the time dependent uptake and retention of radioactivity. This so-called “double mode selection” of the radiotracer by two phenotypic gene expression typical to numerous cancerous cells suggests that [¹¹C]-MTHF can be exploited in PET technology as a valuable addition in PET probe armamentarium. Finally, with very minor modification in the current procedure, the oxidized or reduced derivatives of [¹¹C]-MTHF can be prepared. These include [¹¹C]-leucovorin (folinic acid) and 5-[¹¹C]-methyl-THF, which are both natural metabolites that play a role in cell biology and cancer therapy. Thus, the current preparation of [¹¹C]-MTHF opens the door for the synthesis of a new class of one carbon-11 carriers for different PET applications.

Experimental Section

The synthesis of [¹¹C]-MTHF was carried out on the remotely controlled module (FIG. 9). ¹¹CH₃I was prepared by using cyclotron and GE module was bubbled into TMAO (4 mg) in DMF(300 μl) at −20° C. The temperature of the reaction mixture was then raised to 70° C. in 30 sec and reaction was allowed to take place for 2 min. The reaction was then cooled to room temperature in 30 sec and after mixing with 500 μl water,¹¹CH₂O was reacted with THF (pre-prepared as described in Scheme 2) in the intermediate vial. After 1 min, the reaction mixture was loaded on a SAX cartridge and allowed to pass through under a mild pressure (5-10 psi) of nitrogen gas. The cartridge was then washed with 2 ml of deionized water. The product [¹¹C]-MTHF was eluted from the cartridge by using a sodium phosphate buffer (100 mM, pH 7.4, 4-5 ml) containing 500 mM NaCl and 1% ascorbic acid. Purity and yield of the product was assessed by quantitative analysis on HPLC. Chemical Yield=˜70%.

### Supplemental Materials—Preparation of [¹¹C]-Labeled Precursor for Thymidylate

Please note that the following description refers to FIG. 8. Following preparation of ¹¹CO₂(˜1 Ci at 10 Ci/μmol) at the cyclotron target, ¹¹CH₃I was produced using PETtrace MeI Microlab (GE Medical Systems, Milwaukee, Wis., USA). The ¹¹CH₃I was pushed from the ¹¹CH₃Isynthesis module using helium carrier gas through valve 1 (V1), and was bubbled directly into the reaction vial containing a suspension of TMAO in DMF at −20° C. With V5 in the off position, the pressure inside the vial was maintained via V4 venting to an exhaust bag to collect any escaped radioactivity. After the collection of ¹¹CH₃I was completed, V1 and V4 were closed. The temperature of the reaction vial was raised to 70° C. within 30 seconds, and kept there for 2 minutes to allow complete conversion of ¹¹CH₃I to ¹¹CH₂O. At this stage, the temperature was lowered to 25° C. in 2 minutes, and H₂O was introduced via V2 into the reaction vial to produce an aqueous solution of¹¹CH₂O, which was then transported to the intermediate vial containing THF by opening V5, V6 and V3 and closing V7 to contain the reaction mixture at the intermediate vial. After letting the mixture stand for a minute, the valves V7, V10, and V11 were opened while keeping V6, V8, V9, and V12 in the closed position to allow the mixture to load onto a SAX cartridge, preconditioned with methanol (2 mL) and de-ionized water (5 mL). The pass-through was collected in a waste vial. The cartridge was then washed with H₂O by closing V3, and V7 and opening V8 to manually push H₂O (using a syringe) through the SAX cartridge. The washings were also collected in the waste vial, and the contents of the waste vial were analyzed at the end of washing by placing it in a dose calibrator to count any radioactivity. To elute the ¹¹C-MTHF from the cartridge, V8 and V11 were closed, V9 and V12 were opened, an elution buffer (200 mM sodium phosphate, pH 7.4, containing 500 mM NaCl and 1% ascorbic acid) was manually pushed through the cartridge, and the product was collected in the product vial.

### EXAMPLE 3

### Uptake and Retention in Cancer Cell Lines

Additional studies were performed as described above using the following cell lines:

a. Pancreatic cancer - MIAPaCa and PANC1

b. Lung cancer—H292 and A549

c. Head and Neck cancer—SQ20B, Cal 27 and Fadu

The results provided not only show good uptake and retention of the radioactivity but also cancers from the same organ could be differentiated by uptake enhancement resulting from small dose of methotraxate (MTX- an FDA approved drug that stop endogenous biosynthesis of the MTHF). The clinical implications are for non-invective diagnostics for MTX sensitive and insensitive tumor.

Also animal studies with ¹⁴C-MTHF (nude mice with MIAPaCa tumor) were performed and showed that most of the radioactivity/cell was in the tumor and kidney after one hour.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. The compound [11-¹¹C](R)—N⁵,N¹⁰-methylene-5,6,7,8-tetrahydrofolate (¹¹C-MTHF); [¹¹-¹⁴C]—(R)—N⁵,N¹⁰-methylene-5,6,7,8-tetrahydrofolate (¹⁴C-MTHF); [11C]-leucovorin (folinic acid); [¹⁴C]-leucovorin (folinic acid); 5-[¹¹C]-methyl-THF; 5-[¹⁴C]-methyl-THF; 5-[¹⁴C]-methyl-THF; or a pharmaceutically acceptable salt thereof.

2. A composition comprising the compound of claim 1 and a pharmaceutically acceptable carrier.

3. A pharmaceutical composition comprising a diagnostically effective amount of the compound of claim 1, or a pharmaceutically acceptable salt thereof, together with at least one pharmaceutically acceptable carrier or diluent.

4. A method of detecting a cancer cell comprising contacting the compound of claim 1 to the cell, and imaging the cell by means of positron emission tomography (PET), wherein the compound is employed as a tracer.

5. The method of claim 4, wherein the cell comprises a folate receptor (FR).

6. The method of claim 5, wherein the FR is FR-alpha.

7. A method for in vivo determination of cancer in a subject, comprising visualising potential cancer cells with positron emission tomography (PET), wherein a compound of claim 1 is used as a tracer.

8. A method for in vivo determination of a potential anti-cancer treatment's effectiveness in a subject, comprising i) visualising potential cancer cells with positron emission tomography (PET) or Single Photon Emission Computed Tomography (SPECT), wherein a compound of claim 1 is used as a tracer; ii) administering the potential anti-cancer treatment to the subject; and iii) repeating step i) to determine the effectiveness of the potential anti-cancer treatment

9. A method for the non-invasive determination of the distribution of a tracer compound inside a whole, intact living animal or human body using a physical detection method, wherein the tracer compound is a compound of claim 1.

10. The method of claim 9, wherein the compound is measured by Positron Emission Tomography (PET) or Single Photon Emission Computed Tomography (SPECT).

11. A method of determining the presence of a cell having up-regulated TSase enzyme activity comprising administering the compound of claim 1 to the cell, and imaging the cell by means of positron emission tomography (PET), wherein the compound is employed as a tracer.

12. A method for in vivo imaging comprising the steps of administering to a subject a radiotracer of claim 1, and detecting the radiotracer, wherein the radiotracer is associated with folate receptor (FR).

13. The method of claim 12, further comprising the steps of imaging the subject to detect the distribution of folate receptor.

14. The method of claim 12, further comprising the steps of imaging the subject and analyzing the imaging data.

15. The method of claim 12, wherein the radiotracer is detected by imaging the subject, the method further comprising the steps of administering to the subject a therapeutic agent;
administering to the subject the radiotracer; imaging the subject; and comparing a level of association of the tracer with the folate receptor in the subject before and after administering the therapeutic agent.

16. The method of claim 13, wherein imaging the subject is performed by an imaging modality selected from the group consisting of positron emission tomography, single photon emission computed tomography and combinations thereof.

17. A method for tissue imaging comprising the steps of contacting a tissue that contains folate receptors with a radiotracer of claims 1, and detecting the radiotracer.

18. A method of claim 17, wherein the radiotracer detected in vitro.

19. A method of claim 17, wherein the radiotracer detected ex vivo.
